(19) United States
(12) Patent Application Publication
(10) Pub. No.: US 2005/0282792 A1
(43) Pub. Date: Dec. 22, 2005

(54) Calcitriol/clobetasol propionate compositions for the treatment of psoriasis

(75) Inventor: Philippe Andres, Peymeinade (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA S.A., CHAM (CH)

(21) Appl. No.: 10/942,997

(22) Filed: Sep. 17, 2004

(30) Foreign Application Priority Data
Jun. 17, 2004 (FR) 04/06608

(51) Int. Cl. A61K 31/573

(52) U.S. Cl. 514/179

(57) ABSTRACT

Topically applicable pharmaceutical compositions useful for the treatment of psoriasis contain respective amounts of calcitriol and clobetasol propionate permitting a once-per-day effective regimen of topical application onto the part or parts of the skin affected by psoriasis.

### CROSS-REFERENCE TO PRIORITY APPLICATION

This application claims priority under 35 U.S.C. § 119 of FR 04/06608, filed Jun. 17, 2004, hereby expressly incorporated by reference and assigned to the assignee hereof.

### BACKGROUND OF THE INVENTION

1. Technical Field of the Invention

The present invention relates to pharmaceutical compositions comprising, formulated into a pharmaceutically acceptable medium, calcitriol and clobetasol propionate and to the administration of these compositions for the treatment of psoriasis.

2. Description of Background and/or Related and/or Prior Art

Psoriasis is a chronic inflammatory skin disease which affects approximately 3% of the French population. This disease manifests itself in red plaques covered with whitish squamae which detach from the skin. Psoriasis plaques are often located on the elbows, knees, scalp and lower back, but may extend to all the other parts of the body, such as the face, hands, feet, anatomical folds, nails, etc. Psoriasis may appear at any age, although the first episodes most often arise in the second or third decade. A chronic disease, its development is impossible to predict: phases of recurrence are succeeded by phases of remission. Although this disease rarely endangers a person's life, it nevertheless has a great impact on their quality of life, given its unattractive appearance and its chronic nature. In psoriasis, some people suffer a single psoriasis plaque located in a precise region of the body, while others are subject to extended psoriasis on the whole of the body.

More specifically, the invention described hereinbelow relates to pharmaceutical compositions comprising calcitriol and clobetasol propionate in a specific ratio, such that a synergistic effect between the two active principles is observed in the treatment of psoriasis.

The combination of two or more active principles may prove advantageous, since it allows the doses of the actives that are administered to be reduced and hence makes it possible to reduce the side-effects of these actives. Such combinations are not, however, used conventionally in the treatment of dermatological ailments, given the problems of chemical stability and of interactions between the actives combined in a single formulation.

Calcitriol is the natural and active form of vitamin D. It is used in particular for deficiencies in calcium and to regulate its homeostasis in the body.

Clobetasol propionate, or clobetasol 17-propionate, is a corticosteroid. The mechanism of action of the corticosteroids is attributed to their inhibition of inflammatory processes (Lange, K. et. al., Skin Pharmacol. Appl. Skin Physiol., 13(2): 93-103 (2000)). Throughout the remainder of this specification clobetasol propionate and clobetasol 17-propionate will be used synonymously.

U.S. Pat. No. 4,610,978 describes compositions for topical application in the treatment of dermatological diseases such as, for example, psoriasis, comprising calcitriol. These compositions may further comprise a corticosteroid such as, for example, hydrocortisone or dexamethasone.

WO 00/64450 describes a pharmaceutical composition for dermal use which comprises a vitamin D analogue and a corticosteroid. The examples given relate more particularly to compositions comprising calcipotriol (vitamin D analogue) and betamethasone (corticosteroid). Comparison of measurements of efficacy on patients afflicted by psoriasis for a composition comprising calcipotriol alone, betamethasone alone or the combination of the two actives shows that the effect obtained by the combination corresponds to an additive effect. In the light of this document, therefore, one skilled in the art could not have concluded at all that the combination of a vitamin D analogue with a corticosteroid might exhibit a synergistic effect. Furthermore, this document does not specifically describe the combination of clobetasol propionate with calcitriol.

In FR-2,848,454 the assignee hereof described that the combination of calcitriol with clobetasol propionate made it possible to obtain a synergistic effect in the treatment of certain dermatological ailments such as psoriasis, atopic dermatitis, contact dermatitis and seborrhoeic dermatitis.

### SUMMARY OF THE INVENTION

It has now surprisingly been found that particular amounts of clobetasol propionate and calcitriol provided a particularly advantageous synergistic effect in the treatment of psoriasis, such that the pharmaceutical compositions comprising said compounds are topically applied only once per day to the part or parts of the skin affected by psoriasis.

The present invention accordingly features pharmaceutical compositions for the treatment of psoriasis, comprising clobetasol propionate and calcitriol in amounts appropriate for once-per-day application to the part or parts of the skin affected by psoriasis.

### DETAILED DESCRIPTION OF BEST MODE AND SPECIFIC/PREFERRED EMBODIMENTS OF THE INVENTION

According to one advantageous embodiment of the invention, the subject compositions are more particularly characterized in that they contain from 100 to 700 μg of clobetasol propionate per gram of composition, more particularly from 150 to 500 μg per gram of composition, preferably 500 μg of clobetasol propionate per gram of composition, and more preferably 250 μg of clobetasol propionate per gram of composition.

According to another advantageous embodiment of the invention, the subject compositions are more particularly characterized in that they contain from 0.5 to 10 μg of calcitriol per gram of composition, more particularly from 1.5 to 9 μg per gram of composition, and preferably 3 μg of calcitriol per gram of composition.

Thus, one preferred composition of the invention contains 3 μg of calcitriol per gram of composition and 500 μg of clobetasol propionate per gram of composition.

Another preferred composition of the invention contains 3 μg of calcitriol per gram of composition and 250 μg of clobetasol propionate per gram of composition.

The compositions of the invention further comprise one or more pharmaceutical excipients suitable for topical application.

According to one advantageous embodiment, the composition of the invention is in the form of a gel, cream, ointment, lotion or solution.

Preferably, the creams may be formulated from a mineral oil mixture or from a mixture of beeswax and water which undergoes instantaneous emulsification, to which are added the calcitriol and the clobetasol propionate in solution in a small amount of oil such as almond oil.

Preferably the lotions may be prepared by dissolving calcitriol and clobetasol propionate in a high molecular mass alcohol, such as polyethylene glycol.

Ointments may be formulated by mixing a solution of calcitriol and clobetasol propionate in an oil such as almond oil in heated paraffin, then by leaving the mixture to cool.

Gels may be prepared, preferably, by dispersing or dissolving the calcitriol and the clobetasol propionate in an appropriate ratio in a gel of carbomer, poloxamer or cellulosic type.

Other ingredients may also be added to the topical composition, such as preservatives, for example DL-alpha-tocopherol, or fragrances, if necessary.

The present invention likewise features the formulation of a combination of clobetasol propionate and calcitriol into pharmaceutical compositions useful for the treatment of psoriasis, said medicinal compositions containing an appropriate amount of clobetasol propionate and of calcitriol to be topically applied in a regimen of once per day to the part or parts of the skin affected by psoriasis.

The respective amounts of clobetasol propionate and of calcitriol are as defined above.

Moreover, the medicinal compositions comprise one or more pharmaceutical excipients suitable for topical application and are in the form of a gel, cream, ointment, lotion or solution.

### BRIEF DESCRIPTION OF THE DRAWINGS

FIGS. 1 and 2 represent the mean TSS as a function of time for the seven compositions tested and described in Example 1 below.

FIGS. 3 and 4 represent the number of subjects whose lesion was healed as a function of time by the seven compositions tested and described in Example 1 below.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Evaluation of the Efficacy of Four Different Compositions of the Invention in the “Modified Dumas-Scholtz Test: Psoriasis Treatment in Minizones”

The comparative study described below, a “single-centre, randomized, masked investigator intra-individual, and active-comparator-controlled” study, was conducted in order to evaluate the efficacy and local tolerance of four inventive compositions, comprising different concentrations of clobetasol propionate and calcitriol, by comparison with three compositions not in accordance with the invention.

The four inventive compositions were as follows:

(1) composition containing, respectively, 250 μg of clobetasol propionate and 9 μg of calcitriol per gram of composition, more particularly a cream containing 250 μg/g of clobetasol propionate in combination with an ointment containing 9 μg/g of calcitriol;

(2) composition containing, respectively, 250 μg of clobetasol propionate and 3 μg of calcitriol per gram of composition, more particularly a cream containing 250 μg/g of clobetasol propionate in combination with an ointment containing 3 μg/g of calcitriol (Silkis®);

(3) composition containing, respectively, 500 μg of clobetasol propionate and 9 μg of calcitriol per gram of composition, more particularly a cream containing 500 μg/g of clobetasol propionate (Dermoval®) in combination with an ointment containing 9 μg/g of calcitriol;

(4) composition containing, respectively, 500 μg of clobetasol propionate and 3 μg of calcitriol per gram of composition, more particularly a cream containing 500 μg/g of clobetasol propionate (Dermoval®) in combination with an ointment containing 3 μg/g of calcitriol (Silkis®).

The three compositions not according to the invention were as follows:

(5) composition sold under the name Daivobet®, consisting of an ointment comprising the combination 500 μg/g of betamethasone dipropionate (corticosteroid) and 50 μg/g of calcipotriol as active substances;

(6) composition containing 250 μg of clobetasol propionate per gram of composition in combination with the vehicle of Silkis (ointment combining petroleum jelly and liquid paraffin);

(7) composition containing 500 μg of clobetasol propionate per gram of composition (Dermoval®) in combination with the vehicle of Silkis (petroleum jelly/liquid paraffin ointment).

The objective of the study is to identify and select the concentrations of calcitriol and of clobetasol propionate which exhibit, following application of the treatment for 21 days to 29 subjects afflicted with psoriasis (65.5% men and 34.5% women), an efficacy profile superior to those of a Daivobet® composition and of compositions containing solely clobetasol propionate, the said clobetasol propionate being present in the same concentration as that of the composition comprising a combination of clobetasol propionate and calcitriol.

Methodology:

The seven compositions tested were randomly applied at seven sites to one or more psoriatic plaques of identical severity which were located on the legs (excluding the anterior face of the tibia), on the trunk or on the arms.

The various compositions were applied under normal use conditions, in other words without occlusion, once per day, except on Sunday, for a total of 18 times in all (i.e., 21 days of experimentation).

The compositions were applied as follows: first only clobetasol propionate is applied and, when the latter has penetrated completely, calcitriol or the vehicle of Silkis is applied under the same conditions without a latency period between the two applications.

Clinical evaluations (scores for erythema, infiltration, desquamation and healing) were made at the time of selection of the patients, at their inclusion (baseline) and on days 4, 8,11, 15,18 and 22 of the study. Undesirable events were also recorded.

The principal criterion measured is the area under the curve (AUC) calculated from day 1 to day 22 (final evaluation) on the TSS (total sum score, i.e., sum of the individual scores for erythema, infiltration and desquamation).

Statistical analysis was performed by way of a variance analysis including “subject”, “zone” and “treatment”, followed by a Tukey test for multiple comparisons.

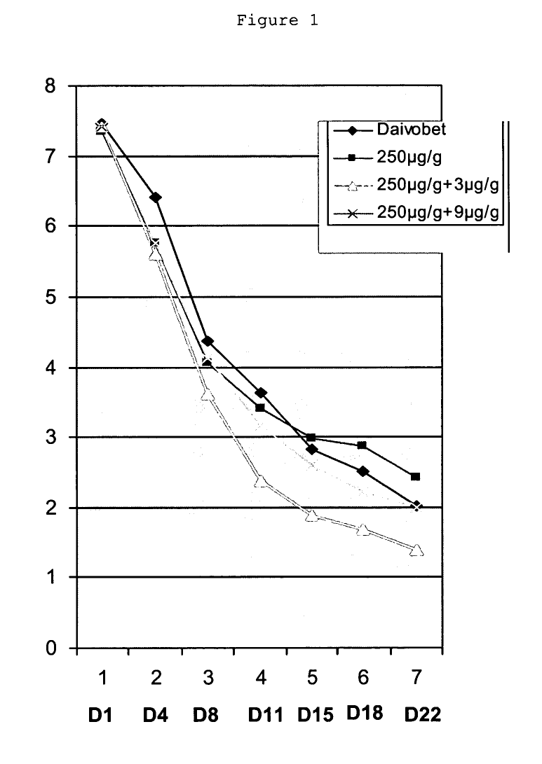

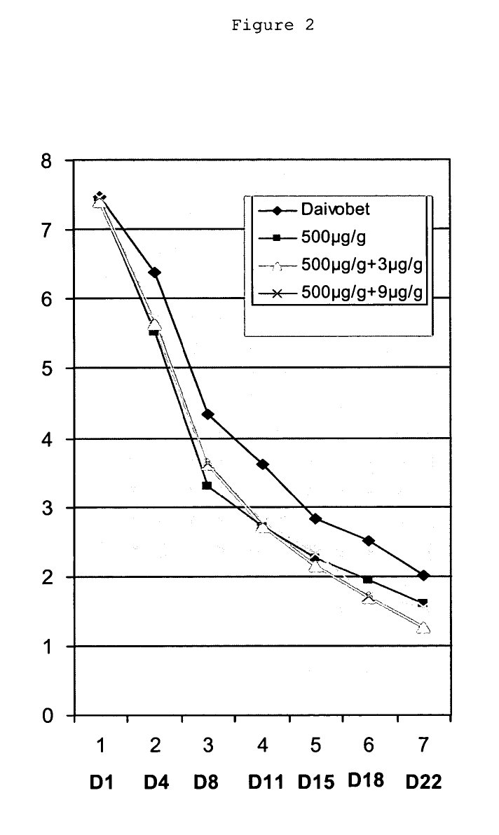

FIGS. 1 and 2 show the mean of the TSS (total sum score=addition of the individual scores for erythema, desquamation and infiltration) as a function of time for seven different, inventive or non-invention compositions which are described in this example.

In FIG. 1 the curve connected by black diamonds corresponds to the treatment with a Daivobet® composition.

The curve connected by dark grey squares corresponds to the treatment with a composition containing 250 μg of clobetasol propionate per gram of composition.

The curve connected by white triangles corresponds to the treatment with a composition of the invention containing, respectively, 250 μg of clobetasol propionate and 3 μg of calcitriol per gram of composition.

The curve connected by black crosses corresponds to the treatment with a composition of the invention containing, respectively, 250 μg of clobetasol propionate and 9 μg of calcitriol per gram of composition.

In FIG. 2 the curve connected by black diamonds corresponds to the treatment with a Daivobet® composition.

The curve connected by dark grey squares corresponds to the treatment with a composition containing 500 μg of clobetasol propionate per gram of composition.

The curve connected by white triangles corresponds to the treatment with a composition of the invention containing, respectively, 500 μg of clobetasol propionate and 3 μg of calcitriol per gram of composition.

The curve connected by black crosses corresponds to the treatment with a composition of the invention containing, respectively, 500 μg of clobetasol propionate and 9 μg of calcitriol per gram of composition.

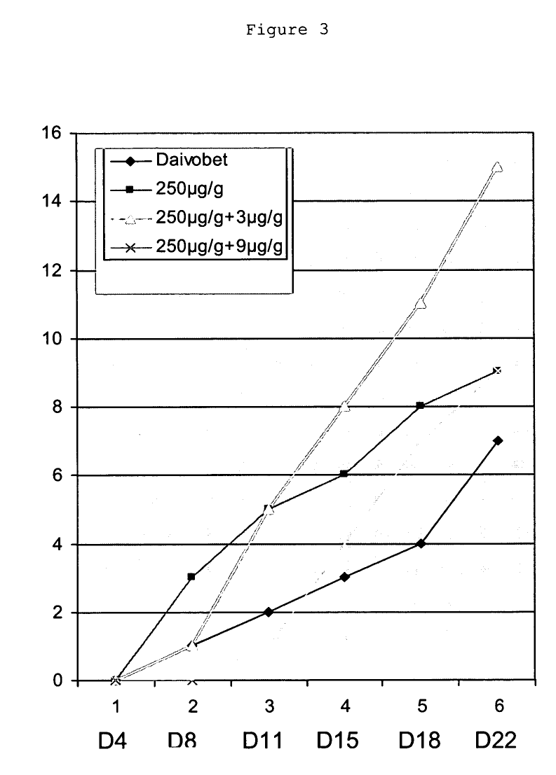

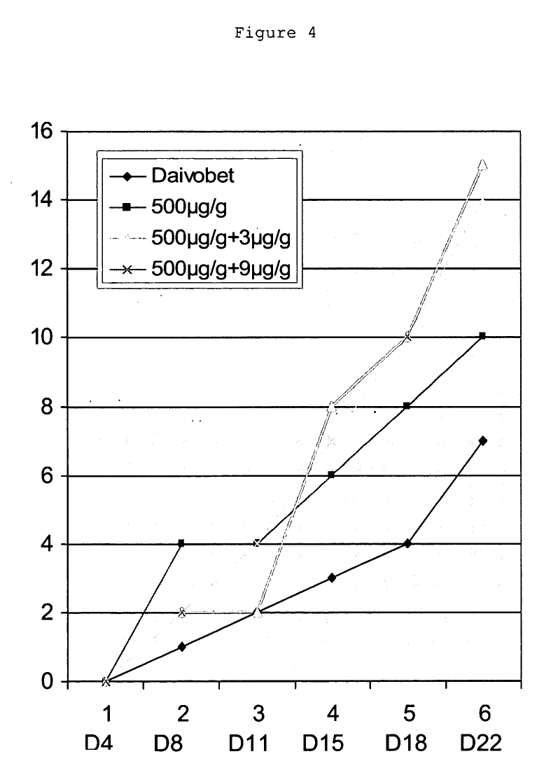

FIGS. 3 and 4 represent the number of subjects whose lesion was healed as a function of time for seven different, inventive or non-invention compositions described in this example.

In FIG. 3 the curve connected by black diamonds corresponds to the treatment with a Daivobet® composition.

The curve connected by dark grey squares corresponds to the treatment with a composition containing 250 μg of clobetasol propionate per gram of composition.

The curve connected by white triangles corresponds to the treatment with a composition of the invention containing, respectively, 250 μg of clobetasol propionate and 3 μg of calcitriol per gram of composition.

The curve connected by black crosses corresponds to the treatment with a composition of the invention containing, respectively, 250 μg of clobetasol propionate and 9 μg of calcitriol per gram of composition.

In FIG. 4 the curve connected by black diamonds corresponds to the treatment with a Daivobet® composition.

The curve connected by dark grey squares corresponds to the treatment with a composition containing 500 μg of clobetasol propionate per gram of composition.

The curve connected by white triangles corresponds to the treatment with a composition of the invention containing, respectively, 500 μg of clobetasol propionate and 3 μg of calcitriol per gram of composition.

The curve connected by black crosses corresponds to the treatment with a composition of the invention containing, respectively, 500 μg of clobetasol propionate and 9 μg of calcitriol per gram of composition.

Results:

29 subjects in total were randomized and included in the study. None of them was excluded from the analyses.

Efficacy: The AUCs of the TSS are illustrated in the Table below.

TABLE

| Compositions tested | AUC of the TSS |
|---|---|
| Clobetasol propionate 250 μg/g + calcitriol 3 μg/g | 66.96 |
| Clobetasol propionate 500 μg/g + calcitriol 3 μg/g | 68.98 |
| Clobetasol propionate 500 μg/g + calcitriol vehicle | 69.60 |
| Clobetasol propionate 500 μg/g + calcitriol 9 μg/g | 71.12 |
| Clobetasol propionate 250 μg/g + calcitriol 9 μg/g | 77.64 |
| Clobetasol propionate 250 μg/g + calcitriol vehicle | 82.65 |
| Daivobet ® | 84.20 |

Conclusion:

The results obtained in terms of AUC of the TSS (primary criterion) for a composition containing clobetasol propionate 250 μg/g and calcitriol 3 μg/g (AUC=66.96) are statistically superior to those obtained for:

a composition containing clobetasol propionate 250 μg/g and a vehicle (AUC=82.65) and for a Daivobet® composition (AUC=84.20).

The same results are observed for the mean AUC of infiltration. In the other criteria the same tendency was observed, without attaining statistical significance. Innocuousness: fourteen undesirable events were reported; none of them was connected to the treatment or led to premature exit from testing.

Under these study conditions a composition containing 250 μg/g of clobetasol propionate and 3 μg/g of calcitriol showed results which were significantly better in efficacy than clobetasol propionate 250 μg/g alone and than Daivobet®. Moreover, this combination showed results of efficacy which were similar to clobetasol propionate 500 μg/g alone (FIGS. 1 and 2).

The combinations of clobetasol propionate 500 μg/g with calcitriol (3 μg/g and 9 μg/g) showed no significant difference in terms of efficacy as compared with clobetasol propionate 500 μg/g alone (see FIG. 2).

All of the seven compositions tested were well tolerated.

The composition containing 250 μg/g of clobetasol propionate and 3 μg/g of calcitriol showed results which were better in terms of healing (see FIGS. 3 and 4) relative:

to clobetasol propionate alone (250 μg/g), to clobetasol propionate 250 μg/g in combination with 9 μg/g of calcitriol, and to Daivobet® (FIG. 3).

The compositions comprising, respectively, a combination:

of 500 μg/g of clobetasol propionate and 3 μg/g of calcitriol and

of 500 μg/g of clobetasol propionate and 9 μg/g of calcitriol allow results to be obtained which are substantially similar to those obtained with 500 μg/g of clobetasol propionate alone and greater than those obtained with Daivobet® (FIG. 4).

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims.

1. A topically applicable pharmaceutical composition useful for the treatment of psoriasis, comprising respective amounts of calcitriol and clobetasol propionate permitting a once-per-day effective regimen of topical application onto the part or parts of the skin affected by psoriasis, formulated into a topically applicable, pharmaceutically acceptable excipient therefor.

2. The pharmaceutical composition as defined by claim 1, comprising from 100 to 700 μg of clobetasol propionate per gram thereof.

3. The pharmaceutical composition as defined by claim 1, comprising from 150 to 500 μg of clobetasol propionate per gram thereof.

4. The pharmaceutical composition as defined by claim 1, comprising 250 μg of clobetasol propionate per gram thereof.

5. The pharmaceutical composition as defined by claim 1, comprising from 0.5 to 10 μg of calcitriol per gram thereof.

6. The pharmaceutical composition as defined by claim 1, comprising from 1.5 to 9 μg of calcitriol per gram thereof.

7. The pharmaceutical composition as defined by claim 1, comprising 3 μg of calcitriol per gram thereof.

8. The pharmaceutical composition as defined by claim 1, comprising 250 μg of clobetasol propionate and 3 μg of calcitriol per gram thereof.

9. The pharmaceutical composition as defined by claim 1, formulated as a gel, cream, ointment, lotion or solution.

10. A dermatological regimen for the treatment of psoriasis, comprising once-per-day topically applying onto the affected area of the skin of an individual afflicted with psoriasis, an effective amount of a pharmaceutical composition which comprises respective amounts of calcitriol and clobetasol propionate permitting said once-per-day topical application, formulated into a topically applicable, pharmaceutically acceptable excipient therefor.

11. The dermatological regimen as defined by claim 10, said pharmaceutical composition comprising from 100 to 700 μg of clobetasol propionate per gram thereof.

12. The dermatological regimen as defined by claim 10, said pharmaceutical composition comprising from 150 to 500 μg of clobetasol propionate per gram thereof.

13. The dermatological regimen as defined by claim 10, said pharmaceutical composition comprising 500 μg of clobetasol propionate per gram thereof.

14. The dermatological regimen as defined by claim 10, said pharmaceutical composition comprising 250 μg of clobetasol propionate per gram thereof.

15. The dermatological regimen as defined by claim 10, said pharmaceutical composition comprising from 0.5 to 10 μg of calcitriol per gram thereof.

16. The dermatological regimen as defined by claim 10, said pharmaceutical composition comprising from 1.5 to 9 μg of calcitriol per gram thereof.

17. The dermatological regimen as defined by claim 10, said pharmaceutical composition comprising 3 μg of calcitriol per gram thereof.

18. The dermatological regimen as defined by claim 10, said pharmaceutical composition comprising 250 μg/g of clobetasol propionate and 3 μg/g of calcitriol.

19. The dermatological regimen as defined by claim 10, said pharmaceutical composition comprising 500 μg/g of clobetasol propionate and 3 μg/g of calcitriol.

20. The dermatological regimen as defined by claim 10, said pharmaceutical composition comprising 500 μg/g of clobetasol propionate and 9 μg/g of calcitriol.
